(19) United States
(12) Patent Application Publication
(10) Pub. No.: US 2010/0069616 A1
(43) Pub. Date: Mar. 18, 2010

(54) ENGINEERED ANTIBODY-NANOPARTICLE CONJUGATES

(75) Inventors: Anna M. Wu, Sherman Oaks, CA (US); Shimon Weiss, Los Angeles, CA (US); Tove Olafsen, Sherman Oaks, CA (US); Fabien Florent Pinaud, Saint Viance (FR); Bhaswati Barat, Derwood, MD (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Regents of the University of California, Oakland, CA (US)

(21) Appl. No.: 12/537,145

(22) Filed: Aug. 6, 2009

Related U.S. Application Data
(60) Provisional application No. 61/086,741, filed on Aug. 6, 2008.

(51) Int. Cl. C07K 16/00

(52) U.S. Cl. 530/391.1; 977/774; 977/742

(57) ABSTRACT

Conjugates of a C-terminal modified diabody and a nanoparticle are provided in which the C-terminal modification introduces a cysteine residue at a C-terminus of the diabody and the diabody is covalently linked to the nanoparticle via a heterobiofunctional linker attached to the introduced cysteine residue.

### CROSS-REFERENCES TO RELATED APPLICATIONS

This application claims priority benefit of U.S. Provisional Application Ser. No. 61/086,741 filed Aug. 6, 2008, the contents of which are incorporated herein by reference in their entirety for all purposes.

### STATEMENT AS TO RIGHTS TO INVENTIONS MADE UNDER FEDERALLY SPONSORED RESEARCH AND DEVELOPMENT

This invention was made with Government support of Grant Nos. CA119367 and EB000312 awarded by the National Institutes of Health. The Government has certain rights in this invention.

### REFERENCE TO A “SEQUENCE LISTING,” A TABLE, OR A COMPUTER PROGRAM LISTING APPENDIX SUBMITTED ON A COMPACT DISK

Not Applicable

### BACKGROUND OF THE INVENTION

In recent years optical imaging has emerged as a sensitive detection method for diagnostic and therapeutic purposes. Quantum dots (Qdots), nanometer scale semiconductor materials, represent an important class of fluorescence probe for biomolecular and cellular imaging (Michalet, X. et al., Science, 307, 538544 (2005)). Qdots are promising as optical probes because they are brighter than traditional organic chromophores, are resistance to photobleaching, have narrow and size-tunable emission wavelength, and have broad excitation spectra. These unique optical properties of Qdots make them appealing as in vivo and in vitro fluorophores in a variety of biological investigations. Furthermore, since the emission wavelength is readily tuned by controlling the size of the Qdots, they can be synthesized to emit different colors, allowing multiplex imaging which is essential in diagnosis of complex biological systems (Xing, Y. et al., Nat. Proc., 2, 1152-1165 (2007)). The use of Qdots as optical probes was originally pioneered by Alivisatos and Weiss and by Nie, in 1998. In the investigations of Alivisatos et al, two different size CdSe-CdS core-shell nanocrystals enclosed in a silica shell were prepared for fluorescent imaging of mouse fibroblast cells (Bruchez, M., Jr. et al., Science, 281, 2013-2016 (1998)). Nie et al investigated receptor mediated endocytosis of transferrin receptor in cultured HeLa cells using CdSe-ZnS Qdots coupled with transferrin (Chan, W. C.; Nie, S., Science, 281, 2016-2018 (1998)). By chemically conjugating antibodies and peptides to their surface, quantum dots can specifically target cellular ligands of interest. Biocompatible Qdots have thus been applied for labeling cells (fixed and live) and tissues (Wu, X. et al., Nat Biotechnol, 21, 41-46 (2003)), long term cell trafficking (Stroh, M. et al., Nat Med, 11, 678-682 (2005)), multicolor cell imaging (Jaiswal, J. K. et al., Nat. Biotechnol., 21, 47-51 (2003)), tumor cell extravasation tracking (Voura, E. B. et al., Nat. Med., 10, 993998 (2004); Tada, H. et al., Cancer Res., 67, 11381144 (2007)), fluorescence resonance energy transfer (FRET)-based sensing (Medintz, L. et al., Nat. Mater., 2, 630-638 (2003)), bioluminescence resonance energy transfer (BRET-based imaging (So, M. K. et al., Nat. Biotechnol., 24, 339-343 (2006)) and sentinel lymph-node mapping (Kim, S. et al., Nat. Biotechnol., 22, 93-97 (2004)). Semiconductor Qdots are also suitable for real-time in vivo imaging (Maysinger, D. et al., Nano Lett. 7(8):2513-20 (2007)). Qdots surface-modified with polyethylene glycol (PEG) were reported to be biocompatible for in vivo cancer targeting and imaging (Maysinger, D. et al., Nano Lett. 7(8):2513-20 (2007); Ballou, B. et al., Bioconjug Chem., IS, 79-86 (2004); Gao, X. et al., Nat. Biotechnol., 22, 969-976 (2004)).

Antibodies can be engineered into a wide variety of formats that retain binding, specificity with target antigen and exhibit optimal properties such as rapid targeting and controlled blood clearance for in vitro or in vivo applications (Kenanova, V.; Wu, A. M. Expert Opin Drug Deliv., 3, 53-70 (2006)). Intact monoclonal antibodies are large (150 kDa) protein molecules. Smaller antibody fragments have been shown to be superior in their ability to extravasate and penetrate solid tumors in vivo, when compared with intact antibodies (Yokota, T. et al., Cancer Res., 52, 34023408 (1992)). Genetically fusing variable light (V_{L}) and heavy (V_{H}) chain domains of a parental antibody through a peptide linker results in the production of a single-chain variable fragments (scFv, 27 kDa), at about ⅙ the size of native antibody, with the same specificity as that of parental antibody. The noncovalent dimers of scFvs are called diabodies (Db, 55 kDa) which can retain full antigen binding activity and specificity in smaller formats (Holliger, P. et al., Proc Natl Acad Sci USA, 90, 6444-6448 (1993)). Our lab has previously demonstrated that radiolabeled diabodies against cancer antigens efficiently targeted to tumors in vivo by microPET (Sundaresan, G. et al., J Nucl Med, 44, 1962-1969 (2003); Wu, A. M. et al., Tumor Targeting, 4, 47-58 (1999)). Their small size (5×7 nm) makes these engineered antibody fragments specifically appropriate for conjugation to nanoscale particles (Carmichael, J. A. et al., Bioconjug Chem., 13, 985-995 (2002)). Conjugation by random chemical modification may be risky for small antibody fragments, due to the possibility of inadvertently disrupting the binding site. Site-specific conjugation is more likely to preserve the binding activity of an antibody. X-ray crystallographic structure of the anti-CEA T84.66 diabody shows that the C-termini of the diabody subunits are almost 70 Angstrom apart and on an alternate face from the antigen combining site (Carmichael, J. A. et al., Bioconjug Chem., 13, 985-995 (2002)). Introduction of cysteine residues at the C-termini of scFv fragment has been considered as an approach to allow site-specific, thiol-reactive coupling at a site away from the antigen binding site to a wide variety of agents (FIG. 1A) (Li, L. et al., Bioconjug Chem., 13, 985-995 (2002); Olafsen, T. et al., Protein Eng Des Sel., 17, 21-27 (2004); Albrecht, H. et al., Bioconjug Chem., 15, 16-26 (2004)) (Sirk, S. unpublished data). Initial work from our laboratory demonstrated site-specific conjugation and radiolabeling of anti-CEA cys-diabody for rapid tumor targeting and imaging in CEA-positive xenograft bearing mouse by microPET (Olafsen, T. et al., Protein Eng Des Sel., 17, 21-27 (2004)).

This invention provides conjugates of cys-diabodies with nanoparticles and methods of using the conjugates in optical imaging for diagnostic purposes. The invention relates to Applicants surprising finding that the conjugates retain their specificities and advantageous affinities for their molecular targets when so used. The Applicants demonstrated that the conjugates retained their dual functionality: antigen binding and fluorescent signaling.

### BRIEF SUMMARY OF THE INVENTION

In a first aspect the invention provides a conjugate of a C-terminal modified diabody and a nanoparticle, wherein the C-terminal modification introduces a cysteine residue at a C-terminus of the diabody (cys-diabody) and the cys-diabody is covalently linked to the nanoparticle by a heterobiofunctional linker attached to the cysteine residue.

In another aspect, the invention provides a method of conjugating a cys diabody to a nanoparticle by 1) making or providing a cysteine modified diabody wherein the modification introduces a cysteine residue at the C-terminus of each monomer of the diabody, wherein the introduced cysteines are joined by disulfide bond between them or may form a disulfide bond with another monomers or another diabody; 2) reducing the disulfide bond to form sulfhydryl groups; and 3) reacting the sulfhydryl groups with a heterobifunctional marker or a maleimide-activated nanoparticle; thereby conjugating the diabody to the nanoparticle. In some embodiments, the nanoparticle is a Quantum rod or carbon nanotube or a Qdot.

In still another aspect, the invention provides a method of detecting a cancer markers on tumor cells by optical imaging by contacting the cancer cell with a conjugate according to the invention. Where the conjugate comprises a fluorescent nanoparticle (e.g., r Qrod), the methods detects the presence of the conjugate by detecting the fluorescence of the conjugated fluorescent nanoparticle. The method may be practiced in vitro or in vivo.

With respect to any of the above aspects, in some embodiments, the nanoparticle is a quantum dot or quantum rod (e.g., a CdSe/ZnS Qdot). In a particular embodiment, the quantum dot is CdSe/ZnS Qdot 655. In a preferred embodiment, the diabody is an anti-cancer antigen diabody. In another preferred embodiment, the quantum dot is a pegylated quantum dot. In a still further embodiment, the quantum dot is PEG Qdot 800. In some embodiments, the conjugate comprises an amine sulfhydryl reactive linker which covalently links the diabody to the nanoparticle. For instance, in some embodiments, the linker is EMCS. In another embodiment, the diabody is linked to the nanoparticle via a heterobifunctional linker which connects the cysteine reside to the quantum dot via an aminopolyethyleneglycol moiety. In still other embodiment, the C-terminal modification of the diabody is an insertion of a Gly-Gly-Cys at the C-terminus of the V_{H} domain of each monomer of the diabody. In yet another embodiment in any of these aspects, the diabody has a pentapeptide sequence Ser-Gly-Gly-Gly-Gly-Gly inserted between the V_{L} and V_{H} domains. In a preferred embodiment, the diabody is an anti-HER2 diabody or an anti-PSCA diabody. The conjugate may comprise a plurality of cys-diabodies covalently linked to the nanoparticle (e.g., 6).

In another set of embodiments with respect to any of the above embodiments, the conjugate comprises an anti-CD20 diabody.

In another aspect still the invention provides conjugates of cys-diabodies as discussed above wherein the cys-diabody is conjugated to a fluorophore other than a nanoparticle. These fluorophore conjugates find diagnostic, therapeutic, and imaging uses as for the nanoparticle conjugates with a cys-diabody. These fluorophore conjugates can be conjugated by heterobifunctional linkers as for the nanoparticles. Suitable cys-diabody conjugates with fluorophores and methods of making the fluorophores are disclosed in Sirk et al., Bioconjug Chem. 2008 December; 19(12):2527-34, the disclosure of which is incorporated herein be reference in its entirety as well as specifically with respect to the fluorophores used in the conjugates, the cys-diabodies of the conjugates, the linkers used, and the particular fluorophore cys-diabody conjugates described therein as well as being incorporated with respect to methods of making the conjugates, the conjugates so made, their methods of using the conjugates, and the experimental data evidencing the construction and operability of the conjugates.

### BRIEF DESCRIPTION OF THE DRAWINGS

FIG. 1. (A) Relative sizes of an intact antibody (IgG) and the engineered antibody fragment, cys-diabody (not to scale). (a) Schematic drawing of an intact Ab showing variable light (V_{L}) and heavy (V_{H}) chain regions and constant (C) regions. (b) Cys-diabody was formed by connecting V_{L} and V_{H} with either 5 or 6 amino acid linker (black line). GGC (black line) was added to the C-termini for conjugation to Qdots. DNA construct and protein are shown. (B) Schematic illustration of the process of conjugating amino PEG Qdot with cys-diabody. EMCS: [N-e-Maleimidocaproyloxy] succinimide ester.

FIG. 2. (A) TEM and HRTEM images of (Left panel) anti-HER2 immunoQdots and (Right panel) Mock conjugated Qdots. (B) Photoluminescence (Emission) spectra of amino PEG Qdot 655 conjugates at excitation wavelength 488 nm. Maximum emission wavelengths are 650.0, 650.5 and 652.5 nm for commercial Qdot 655 (black line), mock conjugated Qdot 655 (blue line) and anti-HER2 immunoQdot 655 (red line) respectively. All spectra are typically around 30 to 50 nm (full width at half maximum).

FIG. 3. Confocal microscopy images of MCF7/HER2 cells. Cells were stained with (A) anti-HER2 immunoQdot 655 and (B) unconjugated Qdot 655. Cell nuclei were counterstained with DAPI and shown in blue. Scale bars: 20 micron.

FIG. 4. Flow cytometry analysis of cys-diabody conjugated Qdot binding with different tumor cells. (A) MCF7/HER2 cells treated with no protein (solid grey), mock conjugated Qdot 655 (dotted black line) and anti-HER2 immunoQdot 655 (solid black line), (B) Binding efficiency of anti-HER2 immunoQdot 655 with different tumor cells. Error bars represent the standard deviation for triplicate flow cytometry experiments. (C) MCF7/HER2 cells treated with (a) mock conjugated Qdot 655 and (b) anti-HER2 immunoQdot 655 and Jurkat cells treated with (c) mock conjugated Qdot 655 and (d) anti-HER2 immunoQdot 655. FL3 (λem: 670 nm long pass) was the filter used for Qdot 655. (D) Competitive cell binding assay by flow cytometry. Anti-HER2 antibody fragment, minibody (Olafsen, T. et al., Cancer Res., 65, 5907-5916 (2005)) was used as competitor. Samples were assayed in triplicate and means±SEM are shown, normalized to the signal obtained in the absence of competitor.

FIG. 5. (A) Cell binding assay of NIR Qdots conjugated cys-diabody. (a) MCF7/HER2 breast cancer cells treated with no protein (solid grey), mock conjugated Qdot 800 (dotted black line) and anti-HER2 immunoQdot 800 (solid black line). (b) LNCaP/PSCA prostate cancer cells treated with no protein (solid grey), mock conjugated Qdot 800 (dotted black line) and anti-PSCA immunoQdot 800 (solid black line). FL5 (λem: 740 long pass) was the filter used for Qdot 800. (B) Normalized emission spectra of amino PEG Qdot 800 conjugates. Excitation wavelength was 532 nm. Corresponding emission peaks and associated full-width half-maximum values were 787.9 and 88.95, 785.7 and 89.19, and 789.0 and 89.62 nm for mock conjugated Qdot 655 (black line), anti-HER2 immunoQdot 800 (red line) and anti-PSCA immunoQdot 800 (brown line) respectively.

FIG. 6. Multicolor QD staining of human prostate cancer cells. (A) Flow cytometry analysis of LNCaP/PSCA cells treated with (a) mock conjugated Qdot 655 and Qdot 800 and (b) anti-HER2 immunoQdot 655 and anti-PSCA immunoQdot 800. (B) In vitro fluorescence imaging of LNCaP/PSCA cells. (a) Cells were stained with (1) mock conjugated Qdot 655 and Qdot 800 and (2) anti-HER2 immunoQdot 655 and anti-PSCA immunoQdot 800. Image was acquired with a filter (550 to 900 nm). (b) Representative fluorescence spectrum of the indicated conjugates obtained from cells. The fluorescence images are raw data from a color CCD camera.

### DETAILED DESCRIPTION OF THE INVENTION

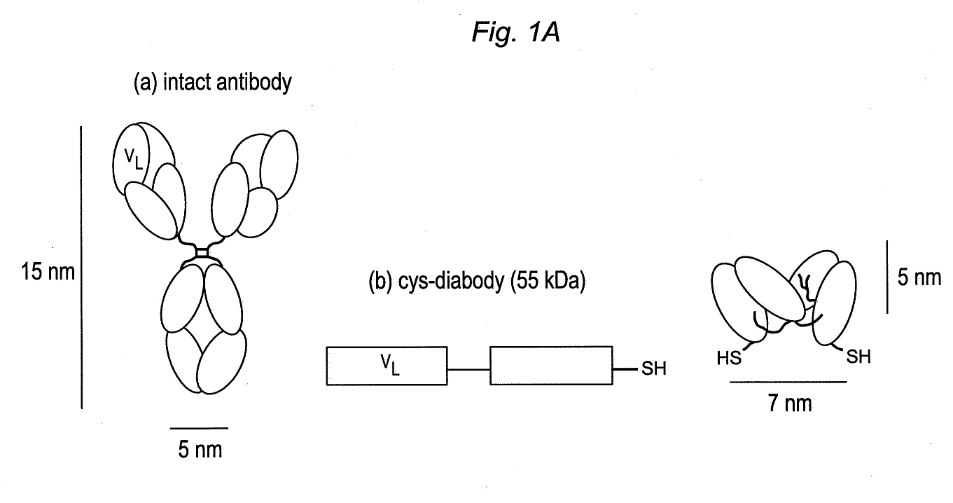

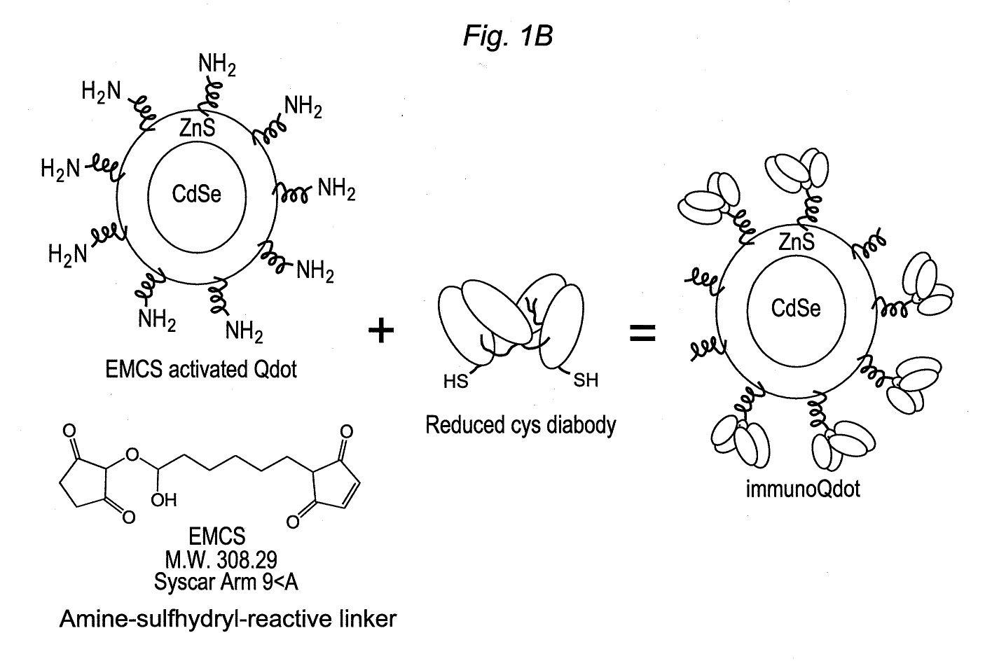

The Her kinase growth factor receptor HER2/neu and prostate stem cell antigen (PSCA) are well characterized cell surface proteins whose expression is elevated in a subset of breast, prostate, and other epithelial cancers. Both proteins are targets for antibody therapeutics. The transmembrane glycoprotein of 185 kDa (p185^{HER-2}), encoded by the HER2/neu proto-oncogene, is overexpressed in 20-30% of breast cancers and in some other cancers. Trastuzumab (Herceptin; Genentech, San Francisco, Calif.) is the humanized version of the 4D5 monoclonal antibody (mAb) that has been approved by the FDA for the treatment of p185^{HER-2} positive tumors. Anti-HER2 cys-diabody was constructed from the variable regions of trastuzumab with the introduction of a cysteine residue at the C-terminal of the diabody. The murine 1G8 (mulG8) mAb directed against PSCA prevents prostate tumor establishment, growth and metastasis in murine models (Safran et al., Proc Nat. Acad Sci USA 98:2658-2663 (2001). Affinity matured recombinant scFv fragment composed of peptide-linked V_{L} and V_{H} domains, derived from the humanized IG8 mAb (2B3) was used as template for anti-PSCA cys-diabody (Lepin, E. unpublished data). For coupling to Qdots or other nanoparticles smaller antibody fragments would be preferable to intact IgGs (FIG. 1A), otherwise the overall size of the antibody-Qdot conjugate becomes quite large.

In the present work, anti-HER2 and anti-PSCA cys-diabodies were site specifically coupled to visible/near infrared (NIR) Qdots and then these immunoQdots were used as targeted optical probes for in vitro cell imaging. Amino PEG CdSe/ZnS Qdot 655 (emission maxima at 655 nm, Invitrogen, Carlsbad, Calif.) was first conjugated to the heterobifunctional cross-linker[N-emaleimidocaproyloxy] succinimide ester (EMCS) (pierce, Rockford, Ill.), yielding a maleimide-nano crystal surface (FIG. 1B). Anti-HER2 cys-diabody was reduced with dithiothreitol (DTT) in parallel. The maleimide-functionalized Qdot 655 was allowed to react with reduced cys-diabody for 1 hour at pH 7.4 and the final conjugate was purified using a 100 kD ultrafiltration unit, Amicon Ultra-4 (Millipore Corp., Bedford, Mass.). The final complex was stored in 10 mM borate buffer, pH 7.4 at 4° C. and was termed as anti-HER2 immunoQdot 655. PSCA antibodies with suitable antigen binding domains are taught in U.S. patent application Ser. No. 10/769,479, filed Jan. 29, 2004, and U.S. patent application Ser. No. 10/769,308, filed Jan. 29, 2004, the contents of which are incorporated by reference with respect to the anti-PSCA antibodies and the antigen binding fragments thereof and further particularly also with respect to the uses of such antibodies and fragments in cancer diagnostics, therapy and imaging.

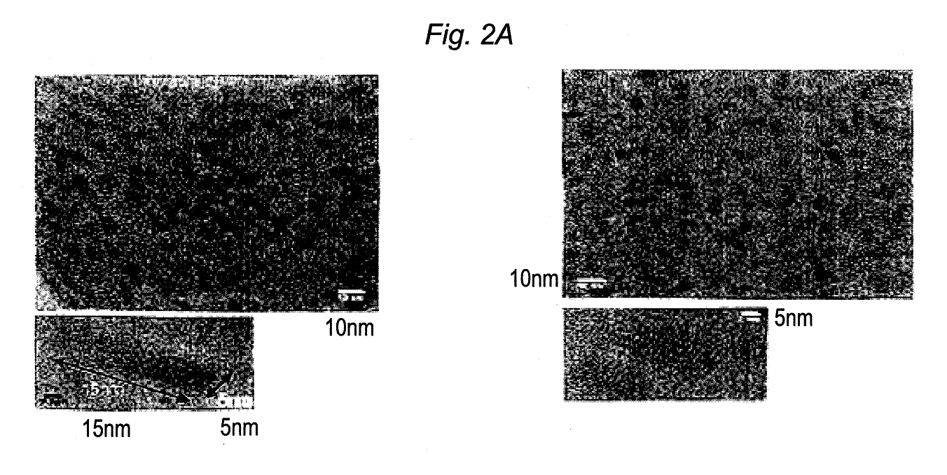

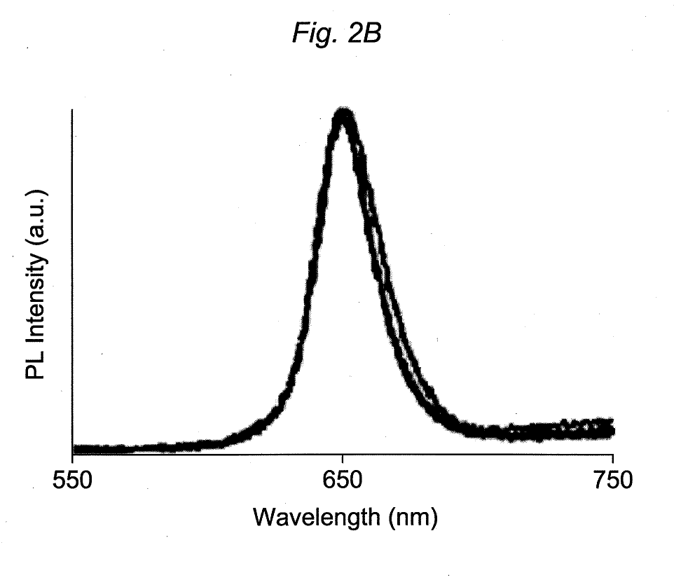

To visualize the structure of synthesized anti-HER2 immunoQdot 655, transmission electron microscopy (TEM) was performed on anti-HER2 immunoQdot 655 and mock conjugated Qdot 655. TEM bright field images revealed that Qdots were uniform in size at approximately 15×5 nm (FIG. 2A).

Photoluminescence (PL) measurements of Qdots were performed by excitation with a 488 nm laser. FIG. 2B shows that the spectrum of anti-HER2 immunoQdot 655 is still symmetric and almost identical to that of commercial Qdots with only a slight blue shift.

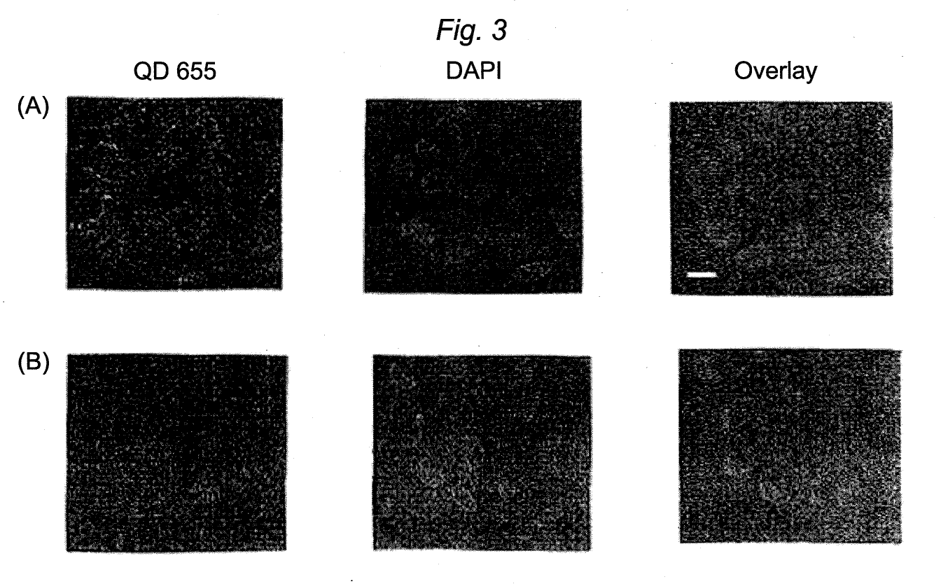

To determine the HER2 receptor binding affinity of anti-HER2 immunoQdot, HER2-transfected human breast carcinoma MCF7/HER2 cells (Olafsen, T. et al., Cancer Res., 65, 5907-5916 (2005)) were incubated with anti-HER2 immunoQdot 655 and examined by confocal microscopy (Carl Zeiss, excitation: Argon Laser 488 nm). The result demonstrated homogeneous surface labeling of cell membrane with minimal cytoplasmic compartment labeling. Little non-specific binding to the cells was observed with mock conjugated Qdot 655 (FIG. 3).

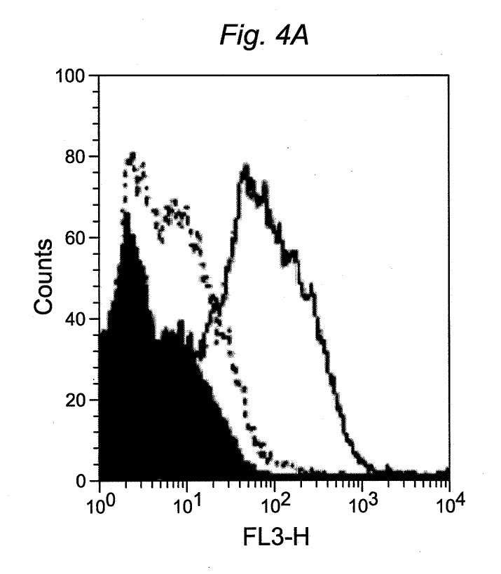

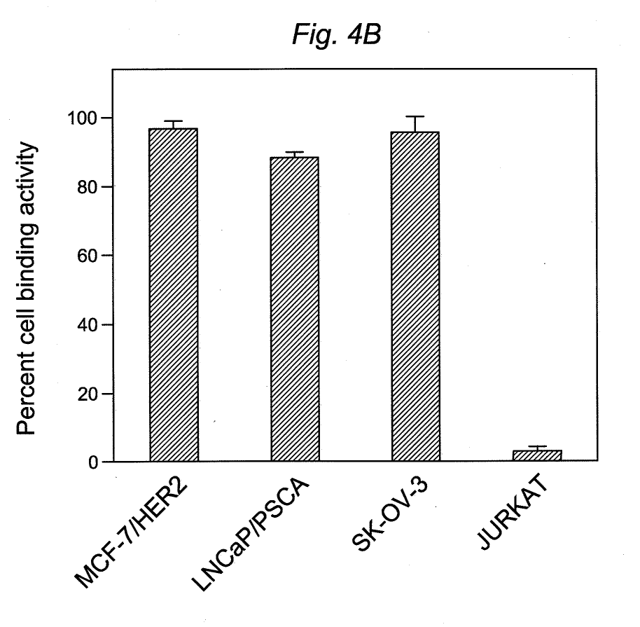

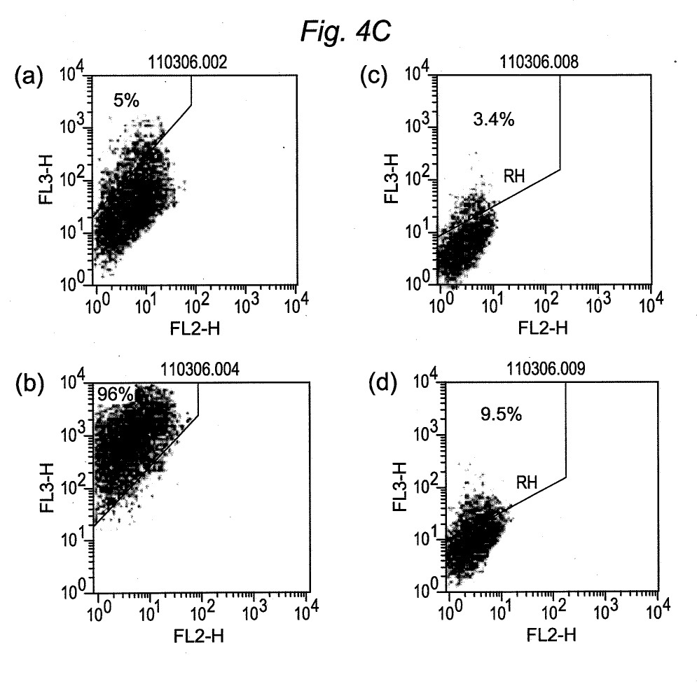

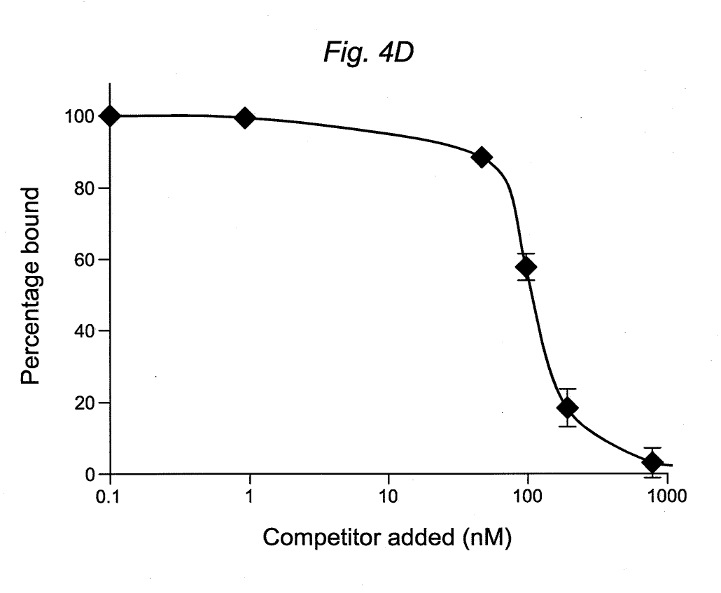

The anti-HER2 immunoQdot 655 was also used to assess HER2 expression on MCF7/HER2 cells by flow cytometry. Results showed a strong fluorescent shift of antiHER2 immunoQdot 655 with MCF7/HER2 cells (FIG. 4A). In parallel, the other control experiments were performed to show the specificity of anti-HER2 immunoQdot 655, i.e. MCF7/HER2 cells binding with mock conjugated Qdot 655 (FIG. 4A) or antiCD20 immunoQdot 655 (irrelevant antibody, negative result) (data not shown). These results clearly demonstrated lack of binding of these non-specific antibodies to HER2 positive cells. Anti-HER2 immunoQdot 655 also bound efficiently to HER2 expressing SK-OV-3 ovarian carcinoma cells and LNCaP/PSCA prostate cancer cells (which also express HER2) (FIG. 4B). No binding was seen to HER2-negative Jurkat cells (FIG. 4C).

Specific binding of anti-HER2 immunoQdot 655 was demonstrated by cell-based competition, in which Qdot conjugated cys diabody was incubated simultaneously in presence of increasing concentrations (0.1-1,000 nM) of competitor and analyzed by flow cytometry (FIG. 4D). This competition study confirmed that anti-HER2 immunoQdot 655 retained the same epitope specificity as that of the anti-HER2 antibody fragment and displayed relative affinity in the nanomolar range.

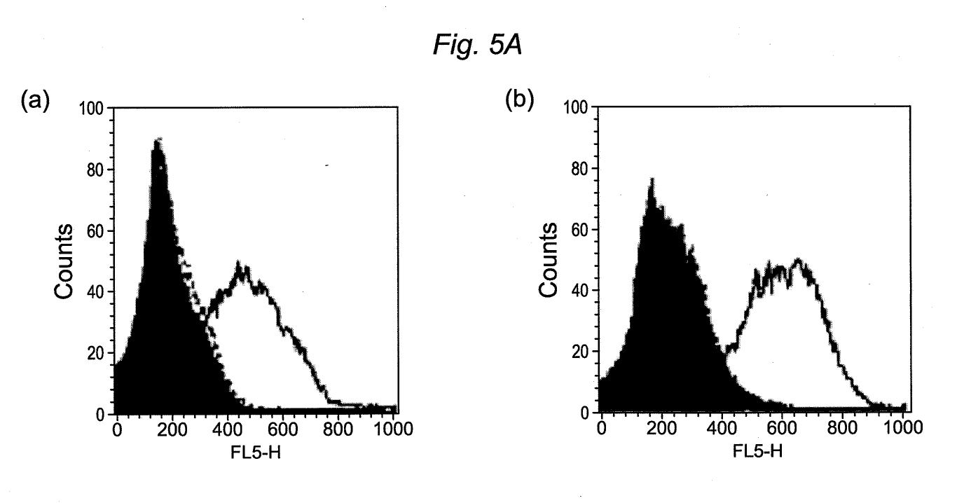

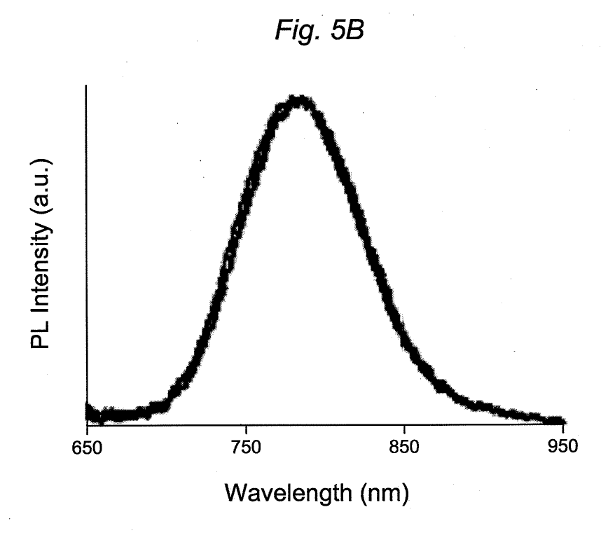

In small animals, NIR (700-900 nm) fluorescence imaging is expected to have major utility, because the absorbance spectra for biomolecules reach minima in the NIR region, providing a window for in vivo optical imaging. We extended the coupling of anti-HER2 cys-diabody to amino PEG CdSe/ZnS Qdot 800 (NIR Qdots, emission maxima at 785 nm, Invitrogen, anti-HER2 immunoQdot 800). The specific binding of anti-HER2 immunoQdot 800 on MCF7/HER2 cells was confirmed by cell binding assay (FIG. 5A (a)). In addition to the anti-HER2 specific antibody fragment, applying the same thiol chemistry we conjugated anti-PSCA cys-diabody with amino PEG Qdot 800 using EMCS (anti-PSCA immunoQdot 800). The result showed strong binding of anti-PSCA immunoQdot 800 with PSCA transfected human prostate cancer LNCaP/PSCA cells 27 (FIG. 5A (b)).

Following excitation with a 532 nm laser, the PL spectrum measurements of the Qdot showed maxima at around 785 nm (FIG. 5B). There was no significant change observed in unconjugated and antibody conjugated Qdot spectra.

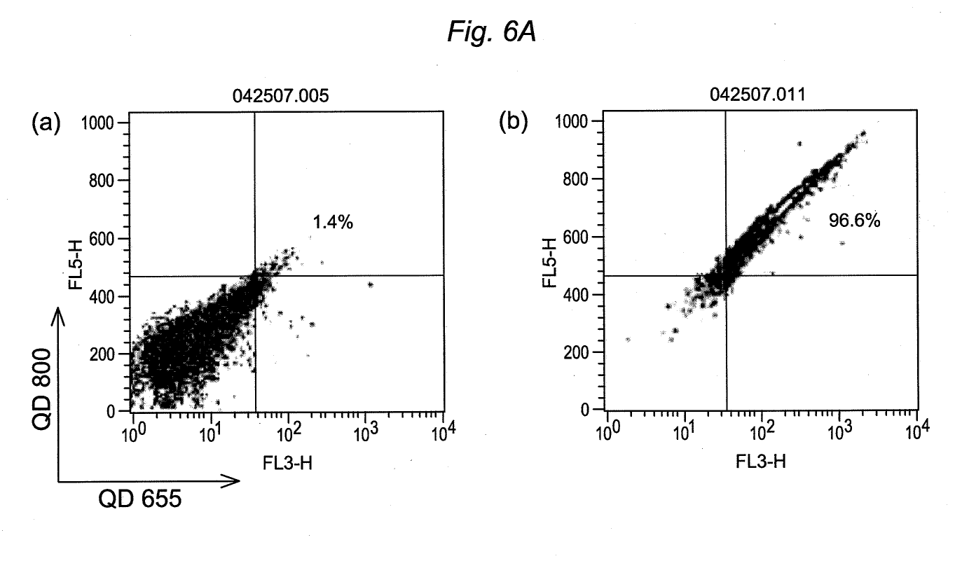

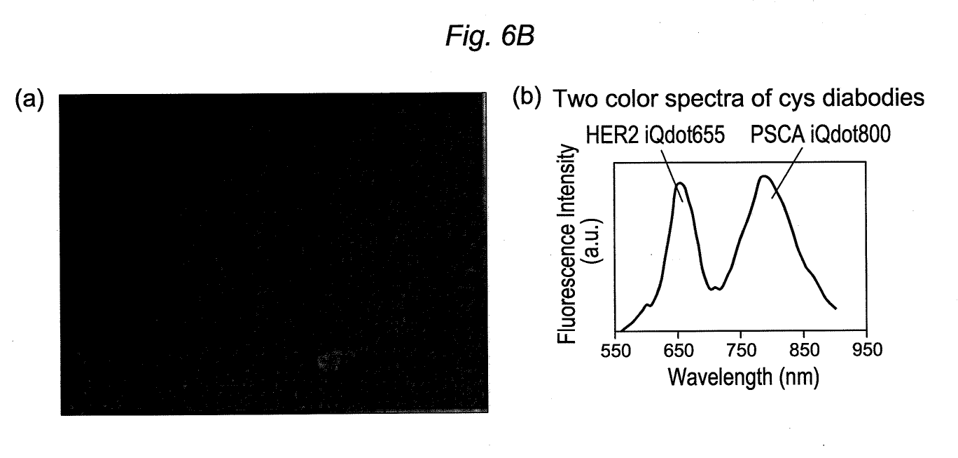

Initially using individual Qdot conjugated cys-diabodies, anti-HER2 immunoQdot 655 and anti-PSCA immunoQdot 800, the expression of each cancer antigen, HER2 and PSCA, was examined on different cancer cells (Supporting information; Table 1). The simultaneous detection of the two cancer markers on LNCaP/PSCA prostate cancer cells (which also express HER2) was then demonstrated using a mixture of two immunoQdots, anti-HER2 immunoQdot 655 and anti-PSCA immunoQdot 800. Flow cytometric analysis showed that 96% of LNCaP/PSCA cells were stained with both immunoQdots, compared to minimum background staining (1.4%) with mock conjugated Qdots (FIG. 6A). To examine the feasibility of multiplex fluorescence imaging, LNCaP/PSCA prostate cancer cells were incubated with two different Qdot conjugates and imaged using a Maestro optical system (CRI, Inc., Woburn, Mass.) (FIG. 6B(a)) The spectral analysis showed the presence of two distinct peaks of anti-HER2 immunoQdot 655 and anti-PSCA immunoQdot 800 (FIG. 6B(b))

In this work, we report the site-specific conjugation of engineered antibody fragments with visible NIR quantum dots for in vitro cell labeling and multiplex imaging. The amine modified quantum dots used in this work include a PEG spacer covalently attached to the Qdot surface. We found that the PEG linker gave less non-specific background compared to the corresponding carboxyl-modified Qdot 655, which does not possess a PEG linker (unpublished data). This characterization is most likely due to the increased hydrophilicity and higher stability resulting from the PEG-coating.

PEGylated Qdots have been previously described for imaging of whole animals (Gao, X. et al., Nat. Biotechnol., 22, 969-976 (2004)). Addition of multiple PEG molecules provides improved biocompatibility and blood retention time. These improved properties of immunoQdots can facilitate their use as optical imaging probes in vivo. Recently the delivery of Qdot 655 labeled antibody to tumor cells was investigated by in vivo real-time tracking (Tada, H. et al., Cancer Res., 67, 1138-1144 (2007)). ROD modified Qdots have also recently been tracked in vasculature by their binding with integrins (Smith, B. R. et al., Nano Lett. (in Press) (2008)).

Most recent studies have been performed using streptavidin conjugated quantum dots to label antigen on the surface of the cells (Fountaine, T. J. et al., Mod Pathol., 19, 1181-1191 (2006); Laiswal, J. K. et al., Nat. Methods., 1, 73-78 (2004); Howarth, M. et al., Proc Natl. Acad Sci USA., 102, 7583-7588 (2005)). In addition, several groups have developed methodologies for introducing specificities onto Qdots by conjugating intact antibodies (Tada, H. et al., Cancer Res., 67, 11381144 (2007); Gao, X. et al., Nat Biotechnol., 22, 969-976 (2004)). One potential shortcoming of the existing Qdot conjugation with biomolecules, especially vis-a-vis in vivo applications, is that the Qdot bioconjugates become quite large (˜40-50 nm), once streptavidin or intact antibodies are incorporated. For large nanoparticles, it would be difficult to traverse the endothelium and penetrate into tissues and tumors. In contrast, in this work, small antibody fragments, cys-diabodies were directly labeled to Quantum dots. The overall small size (approximately 15-20 nm) of these immunoQdots make them ideal candidate for application in living organisms.

In conclusion, cys-diabodies are small, bivalent tumor-targeting antibody fragments that retain antigen binding specificity after incorporation of the cysteine modification at the C-termini. Their small size (5×7 nm) and favorable pharmacokinetics make them ideal for use in imaging and therapeutic applications. The present work demonstrates site-directed thiol-specific conjugation of cys-diabodies at a site away from the antigen binding site to the commercially available amino PEG quantum dots. The immunoQdots retain the photoluminescence properties of the unconjugated Qdots as well as the antigen binding specificity. The overall small size of cys-diabody conjugated Qdots should be suitable for use in biological applications. The results of Qdot conjugation to cys-diabodies with different tumor specificities opens up new prospects for multiplex imaging in cancer. This thiol-reactive conjugation approach can be used as a generalized platform for site-specific coupling of cys-diabodies with a wide variety of other nanoparticles, such as Quantum rods or carbon nanotubes.

This work demonstrates successful thiol-specific, oriented coupling of tumor targeting small engineered antibody fragments, cys-diabodies, at a position away from the antigen binding site. These bioconjugated quantum dots (termed immunoQdots) demonstrated dual functionality: retention of antigen binding as well as fluorescent signal. Simultaneous detection of two tumor antigens on LNCaP/PSCA prostate cancer cells (which express PSCA and HER2) in culture was possible using two immunoQdots, anti-HER2 immunoQdot 655 and anti-PSCA immunoQdot 800. The Applicants work in this field has now been published. See, Barat et al., Bioconjug Chem. 2009 Jul. 31 (epublished), the disclosures of which is incorporated herein be reference in its entirety as well as specifically with respect to the fluorophores used in the conjugates, the cys-diabodies of the conjugates, the linkers used, and the particular fluorophore cys-diabody conjugates described therein as well as with respect to methods of making the conjugates, the conjugates so made, their methods of use, and the experimental data evidencing their construction and operability.

### EXAMPLES

### Example 1

### Design, Expression and Purification of Cys Diabodies

The anti-HER2 diabody was constructed from trastuzumab (Herceptin™) human variable regions using an existing single-chain variable fragment (scFv) gene construct as template (Olafsen et al., Cancer Res., 65: 5907-5916 (2005)). Anti-HER2CysDb was constructed from an existing minibody, composed of two trastuzumab (Herceptin™, Genentech) humanized scFvs linked to the C_{H}3 domain of human IgG1. The scFv orientation and linker of the anti-HER2 minibody were as follows: V_{L}-GSTSGGGSGGGSGGGGSS-V_{H}. Overlapping PCR was used to shorten the 18 amino-acid-linker in the anti-HER2 scFv gene with a 5 amino-acid-linker (SGGGG). A Gly-Gly-Cys modification at the C-terminus of the VH domain in the pEE12 expression vector was also used (Lonza Biologics, Slough, UK) (Sirk, S. unpublished data), The pEE12 construct contains a mammalian leader sequence for extra cellular expression of the recombinant protein.

For anti-HER2 cys-diabody, 2.5×106 NSO cells (Galfre G. et al; Methods Enzymol. 1981, 73:3-46) were transfected by electroporation with 10 micrograms of linearized plasmid DNA and selected in glutamine-deficient media as described (Yazaki et al., Immunol Methods., 253:195-208 (2001)). Anti-HER2 cys-diabody, expression was screened by SDS-PAGE using pre-cast 4-20% gels (Bio-Rad, Hercules, Calif.), under reducing and non-reducing conditions. The highest expressing clones were expanded into triple flasks (Nunclon, Rochester, N.Y.). Supernatants containing the anti-HER2 cys-diabody were loaded onto a Protein L column (Pierce, Rockford, Ill.). Bound protein was eluted using 0-100% gradient of 0.1 M glycine (pH 2.5) in PBS (pH 7.0). Eluted fractions were collected in the presence of 1/10 volume of 2 M Tris HCl pH 8.0. Eluted fractions containing the desired protein were pooled, dialyzed against PBS and concentrated by Centriprep 30 (Millipore Corp., Bedford, Mass.).

An anti-PSCA diabody was constructed from an existing affinity matured scFv (2B3, human variable regions of antibody against PSCA gene construct, (Olafsen et al., J Immunother, 30:396-405 (2007)). PCR overlap extension was used to amplify the V_{L} and V_{H} domains separately, inserting overlapping 6-amino acid linker (VL-SGGGGS-VH), as well as a Gly-Gly-Cys modification at the C-terminus of the VH domain. The final PCR product of anti-PSCA cys diabody was cloned into pSyn1 bacterial expression vector.

For bacterial expression, Escherichia coli BL21 cells were grown in Luria-Bertani broth (LB) to an OD600 of 0.7, induced with a final concentration of 1 mM IPTG and grown 4 hours at 37° C. Periplasmic extracts were prepared using Peripreps Periplasting Kits (Epicentre, Madison, Wis.). The anti PSCA cys-diabody was purified by immobilized Protein L chromatography as per manufacturer instructions (Pierce).

### Example 2

### Coupling of Qdots to Tumor-Specific Cys Diabodies

Qdots were conjugated to cys diabodies with Qdot 655 or Qdot 800 amino (PEG) quantum dots (Quantum Dot Corp., Hayward, Calif.). Qdots were activated with the heterobifunctional cross-linker [N-e-maleimidocaproyloxy] succinimide ester (EMCS) (Pierce) for 30 minutes at room temperature, yielding a maleimide-nanocrystal surface. Excess EMCS was removed by desalting column. cys diabodies were simultaneously reduced by incubating in 20 mM DTT at room temperature for 30 min. Then, activated Qdots were covalently coupled with reduced antibody fragment at room temperature for one hour in borate buffer (pH 7.4). The molar ratio of antibody fragment to the Qdots was 22:1. The reaction was quenched by adding 34 micrograms of N-ethyl maleimide (NEM) (Pierce) per mg of antibody fragment. The uncoupled free cys diabody and excess NEM were removed by three washes using a 100 KD ultrafiltration unit, Amicon Ultra-4 (Millipore Corp.) The final complex was kept in 10 mM borate buffer at 4° C.

### Flow Cytometry

Human breast tumor cell line, MCF7/HER2 was incubated with either anti-HER2 immunoQdot 655 or anti-HER2 immunoQdot 800 for 1 hour at 4° C. in PBS containing 1% BSA. Prostate cancer cells LNCaP/PSCA was incubated with anti-PSCA immunoQdot 800 using the same condition. Antibody fragments binding to tumor cells were quantified by FACS Calibur flow cytometer (Beckton Dickinson, UK) and data were analyzed by Cell Quest software. FL3 (λₑₘ: 670 run long pass) and FL5 (λₑₘ: 740 run long pass) were the filters used for Qdot 655 and Qdot 800 respectively.

### Confocal Microscopy

MCF7/HER2 cells were plated on poly-L lysine coated glass coverslips (BD Biosciences, San Jose, Calif.) in 12 well-plates in DMEM medium containing 5% Fetal bovine serum (FBS) for 24 hours. The next day, cells were incubated with mock conjugated Qdot 655 and anti-HER2 immunoQdot 655 in PBS/1% FBS on ice for 1 hr. Cells were then fixed with 3.7% paraformaldehyde at 4° C. for 30 min. Cell nuclei were counterstained with DAPI. Coverslips were mounted on glass slides and observed using a Leica TCS—SP inverted confocal microscope equipped with a 100× oil immersion objective lens.

TABLE 1
Binding assay of different immunoQdots
with different tumor cell lines

| | Anti-HER2 | Anti-PSCA |
|---|---|---|
| Cell line | immunoQdot 655 | immunoQdot 800 |
| Jurkat | − | − |
| MC7/HER2 | + | − |
| SKW | − | + |
| LNCaP/PSCA | + | + |

All publications, patents, patent applications, and/or other documents cited in this application are incorporated by reference in their entirety, to the extent not inconsistent with the present disclosure, for all purposes to the same extent as if each individual publication, patent, patent application, and/or other document were individually indicated to be incorporated by reference for all purposes.

A2 anti-PSCA cys-diabody nucleic acid and protein sequences.

DNA
GACATTCAGCTGACCCAGTCCCCAAGCTCTTTGTCCGCCTCTGTGGGGGA
TAGGGTCACCATCACCTGCAGTGCCAGTTCAAGTGTAAGATTCATTCACT
GGTACCAGCAGAAACCAGGAAAAGCTCCCAAAAGACTCATCTATGACACA
TCCAAACTGGCTTCTGGCGTCCCTTCTAGGTTCAGTGGCTCCGGGTCTGG
GACAGACTTCACCCTCACCATTAGCAGTCTGCAGCCGGAAGATTTCGCCA
CCTATTACTGTCAGCAGTGGGGTAGCAGCCCATTCACGTTCGGACAGGGG
ACCAAGGTGGAGATAAAAGGTGGTGGTGGTTCGGAGGTTCAGCTGGTGGA
GTCTGGGGGTGGTCTTGTGCAGCCAGGGGGCTCACTCCGTTTGTCCTGCG
CAGCTTCTGGCTTCAACATTAAAGACTACTATATACACTGGGTGCGTCAG
GCCCCTGGTAAGGGCCTGGAATGGGTTGCATGGATTGATCCTGAGTACGG
TGACTCTGAATTTGTCCCGAAGTTCCAGGGCCGGGCCACTATGAGCGCAG
ACACATCCAAAAACACAGCCTACCTGCAGATGAACAGCCTGCGTGCTGAG
GACACTGCCGTCTATTATTGTAAGACGGGGGGTTTCTGGGGTCGTGGAAC
CCTGGTCACCGTCTCGAGCGGTGGATGT
Protein
DIQLTQSPSSLSASVGDRVTITCSASSSVRFIHWYQQKPGKAPKRLIYDT
SKLASGVPSRFSGSGSGTDFTLTISSLQPEDFATYYCQQWGSSPFTFGQG
TKVEIKGGGGSEVQLVESGGGLVQPGGSLRLSCAASGFNIKDYYIHWVRQ
APGKGLEWVAWIDPEYGDSEFVPKFQGRATMSADTSKNTAYLQMNSLRAE
DTAVYYCKTGGFWGRGTLVTVSSGGC

DNA and protein sequences for anti-CD20 cysdiabody scFV subunit. The sequence begins with the mammalian leader sequence (bold type) followed by the Vl domain, 5-amino acid linker domain (underlined) VH domain and C-terminal cysteine modification (bold type):

```
atggattttcaggtgcagattatcagcttcctgctaatcagtgcttcagtcataatgtcc
  M D F Q V Q I I S F L L I S A S V I M S
agaggacaaattgttctctcccagtctccagcaatcctgtctgcatctccaggggagaag
  R G Q I V L S Q S P A I L S A S P G E K
gtcacaatgacttgcagggccagctcaagtgtaagttacatccactggttccagcagaag
  V T M T C R A S S S V S Y I H W F Q Q K
ccaggatcatcccccaaaccctggatttatgccacatccaacctggcttctggagtccct
  P G S S P K P W I Y A T S N L A S G V P
gttcgcttcagtggcagtgggtctgggacctcttactctctcacaatcagcagagtggag
  V R F S G S G S G T S Y S L T I S R V E
gctgaagatgctgccacttattactgccagcagtggactagtaacccacccacgttcgga
  A E D A A T Y Y C Q Q W T S N P P T F G
ggggggaccaagctggaaataaaaagtggaggcggtggacaggtacaactgcagcagcct
  G G T K L E I K S G G G G G Q V Q L Q QP
ggggctgagctggtgaagcctggggcctcagtgaagatgtcctgcaaggcttctggctac
  G A E L V K P G A S V K M S C K A S G Y
acatttaccagttacaatatgcactgggtaaaacagacacctggtcggggcctggaatgg
  T F T S Y N M H W V K Q T P G R G L E W
attggagctatttatccaggaaatggtgatacttcctacaatcagaagttcaaaggcaag
  I G A I Y P G N G D T S Y N Q K F K G K
gccacattgactgcagacaaatcctccagcacagcctacatgcagctcagcagcctgaca
  A T L T A D K S S S T A Y M Q L S S L T
tctgaggactctgcggtctattactgtgcaagatcgacttactacggcggtgactggtac
  S E D S A V Y Y C A R S T Y Y G G D W Y
ttcaatgtctggggcgcagggaccacggtcaccgtctctgcagga [symbol] tagtag
  F N V W G A G T T V T V S A G [symbol] [symbol] [symbol]       - -
```

### The Sequence for the Her Cys Db Nucleic Acid and Diabody Follows:

```
gatatccagatgacccagtccccgagctccctgtccgcctctgtgggcgatagggtcacc
  D I Q M T Q S P S S L S A S V G D R V T
atcacctgccgtgccagtcaggatgtgaatactgctgtagcctggtatcaacagaaacca
  I T C R A S Q D V N T A V A W Y Q Q K P
ggaaaagctccgaaactactgatttactcggcatccttcctctactctggagtcccttct
  G K A P K L L I Y S A S F L Y S G V P S
cgcttctctggttccagatctgggacggatttcactctgaccatcagcagtctgcagccg
  R F S G S R S G T D F T L T I S S L Q P
gaagacttcgcaacttattactgtcagcaacattatactactcctcccacgttcggacag
  E D F A T Y Y C Q Q H Y T T P P T F G Q
ggtaccaaggtggagatcaaatccggtgggggcggcgaggttcagctggtggagtctggc
  G T K V E I K S G G G G E V Q L V E S G
ggtggcctggtgcagccagggggctcactccgtttgtcctgtgcagcttctggcttcaac
  G G L V Q P G G S L R L S C A A S G F N
attaaagacacctatatacactgggtgcgtcaggccccgggtaagggcctggaatgggtt
  I K D T Y I H W V R Q A P G K G L E W V
gcaaggatttatcctacgaatggttatactagatatgccgatagcgtcaagggccgtttc
  A R I Y P T N G Y T R Y A D S V K G R F
actataagcgcagacacatccaaaaacacagcctacctgcagatgaacagcctgcgtgct
  T I S A D T S K N T A Y L Q M N S L R A
gaggacactgccgtctattattgttctagatggggaggggacggcttctatgctatggac
  E D T A V Y Y C S R W G G D G F Y A M D
tactggggtcaaggaaccctggtcaccgtctcgagtggaggcggttgc
  Y W G Q G T L V T V S S G G G C
```

1. A conjugate of a C-terminal modified diabody and a nanoparticle, wherein the C-terminal modification introduces a cysteine residue at a C-terminus of the diabody and the diabody is covalently linked to the nanoparticle via a heterobifunctional linker attached to the introduced cysteine residue.

2. The conjugate of claim 1, wherein the nanoparticle is a quantum dot.

3. The conjugate of claim 2, wherein the quantum dot is a CdSe/ZnS Qdot.

4. The conjugate of claim 3, wherein the quantum dot is CdSe/ZnS Qdot 655.

5. The conjugate of claim 1, wherein the diabody is an anti-cancer antigen diabody.

6. The conjugate of claim 2, wherein the quantum dot is a pegylated quantum dot.

7. The conjugate of claim 6, wherein the quantum dot is PEG Qdot 800.

8. The conjugate of claim 1, wherein the linker is an amine sulfhydryl reactive linker.

9. The conjugate of claim 2, wherein the linker is EMCS.

10. The conjugate of claim 1, wherein the C-terminal modification is an insertion of a Gly-Gly-Cys at the C-terminus of the V_{H} domain of each monomer of the diabody.

11. The conjugate of claim 10, wherein the diabody has a pentapeptide sequence Ser-Gly-Gly-Gly-Gly (SEQ ID NO:7) inserted between the V_{L} and V_{H} domains.

12. The conjugate of claim 2, wherein the diabody is linked to quantum dot via a heterobifunctional linker which connects the cysteine reside to the quantum dot via an amino polyethyleneglycol moiety.

13. The conjugate of claim 5, wherein the diabody is an anti-HER2 diabody or an anti-PSCA diabody.

14. The conjugate of claim 1, wherein the nanoparticle is a carbon nanotube.

15. The conjugate of claim 1, herein the nanoparticle is a Quantum rod.

16. A method of conjugating a cys diabody to a nanoparticle, said method comprising the steps of:
making a cysteine modified diabody wherein the modification introduces a cysteine residue at the C-terminus of each monomer of the diabody, wherein the diabody the introduced cysteines are joined by a disulfide bond between them;
reducing the disulfide bond to form sulfhydryl groups; and
reacting the sulfhydryl groups with a maleimide-activated nanoparticle; thereby conjugating the diabody to the nanoparticle.

17. The method of claim 16, wherein the nanoparticle is a quantum dot, a Quantum rod or a carbon nanotube.

18. The method of claim 16, wherein the nanoparticle is a Qdot.


SEQUENCE LISTING (9 sequences)

SEQ ID NO: 1 - DNA, 678 bp, Artificial Sequence
  synthetic anti-PSCA cys-diabody A2
gacattcagctgacccagtccccaagctctttgtccgcctctgtgggggatagggtcaccatcacctgcagtgccagttcaagtgtaagattcattcactggtaccagcagaaaccaggaaaagctcccaaaagactcatctatgacacatccaaactggcttctggcgtcccttctaggttcagtggctccgggtctgggacagacttcaccctcaccattagcagtctgcagccggaagatttcgccacctattactgtcagcagtggggtagcagcccattcacgttcggacaggggaccaaggtggagataaaaggtggtggtggttcggaggttcagctggtggagtctgggggtggtcttgtgcagccagggggctcactccgtttgtcctgcgcagcttctggcttcaacattaaagactactatatacactgggtgcgtcaggcccctggtaagggcctggaatgggttgcatggattgatcctgagtacggtgactctgaatttgtcccgaagttccagggccgggccactatgagcgcagacacatccaaaaacacagcctacctgcagatgaacagcctgcgtgctgaggacactgccgtctattattgtaagacggggggtttctggggtcgtggaaccctggtcaccgtctcgagcggtggatgt

SEQ ID NO: 2 - protein, 226 aa, Artificial Sequence
  synthetic anti-PSCA cys-diabody A2
DIQLTQSPSSLSASVGDRVTITCSASSSVRFIHWYQQKPGKAPKRLIYDTSKLASGVPSRFSGSGSGTDFTLTISSLQPEDFATYYCQQWGSSPFTFGQGTKVEIKGGGGSEVQLVESGGGLVQPGGSLRLSCAASGFNIKDYYIHWVRQAPGKGLEWVAWIDPEYGDSEFVPKFQGRATMSADTSKNTAYLQMNSLRAEDTAVYYCKTGGFWGRGTLVTVSSGGC

SEQ ID NO: 3 - DNA, 780 bp, Artificial Sequence
  synthetic anti-CD20 cys-diabody scFv subunit
atggattttcaggtgcagattatcagcttcctgctaatcagtgcttcagtcataatgtccagaggacaaattgttctctcccagtctccagcaatcctgtctgcatctccaggggagaaggtcacaatgacttgcagggccagctcaagtgtaagttacatccactggttccagcagaagccaggatcatcccccaaaccctggatttatgccacatccaacctggcttctggagtccctgttcgcttcagtggcagtgggtctgggacctcttactctctcacaatcagcagagtggaggctgaagatgctgccacttattactgccagcagtggactagtaacccacccacgttcggaggggggaccaagctggaaataaaaagtggaggcggtggacaggtacaactgcagcagcctggggctgagctggtgaagcctggggcctcagtgaagatgtcctgcaaggcttctggctacacatttaccagttacaatatgcactgggtaaaacagacacctggtcggggcctggaatggattggagctatttatccaggaaatggtgatacttcctacaatcagaagttcaaaggcaaggccacattgactgcagacaaatcctccagcacagcctacatgcagctcagcagcctgacatctgaggactctgcggtctattactgtgcaagatcgacttactacggcggtgactggtacttcaatgtctggggcgcagggaccacggtcaccgtctctgcaggaggcggttgctagtag

SEQ ID NO: 4 - protein, 258 aa, Artificial Sequence
  synthetic anti-CD20 cys-diabody scFv subunit
MDFQVQIISFLLISASVIMSRGQIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKSGGGGQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAGGGC

SEQ ID NO: 5 - DNA, 708 bp, Artificial Sequence
  synthetic anti-HER cys-diabody (Cys Db)
gatatccagatgacccagtccccgagctccctgtccgcctctgtgggcgatagggtcaccatcacctgccgtgccagtcaggatgtgaatactgctgtagcctggtatcaacagaaaccaggaaaagctccgaaactactgatttactcggcatccttcctctactctggagtcccttctcgcttctctggttccagatctgggacggatttcactctgaccatcagcagtctgcagccggaagacttcgcaacttattactgtcagcaacattatactactcctcccacgttcggacagggtaccaaggtggagatcaaatccggtgggggcggcgaggttcagctggtggagtctggcggtggcctggtgcagccagggggctcactccgtttgtcctgtgcagcttctggcttcaacattaaagacacctatatacactgggtgcgtcaggccccgggtaagggcctggaatgggttgcaaggatttatcctacgaatggttatactagatatgccgatagcgtcaagggccgtttcactataagcgcagacacatccaaaaacacagcctacctgcagatgaacagcctgcgtgctgaggacactgccgtctattattgttctagatggggaggggacggcttctatgctatggactactggggtcaaggaaccctggtcaccgtctcgagtggaggcggttgc

SEQ ID NO: 6 - protein, 236 aa, Artificial Sequence
  synthetic anti-HER cys-diabody (Cys Db)
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKSGGGGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGC

SEQ ID NO: 7 - protein, 5 aa, Artificial Sequence
  synthetic diabody pentapeptide linker between V-L and V-H domains
SGGGG

SEQ ID NO: 8 - protein, 18 aa, Artificial Sequence
  synthetic anti-HER2 scFv minibody linker between V-L and V-H domains
GSTSGGGSGGGSGGGGSS

SEQ ID NO: 9 - protein, 6 aa, Artificial Sequence
  synthetic anti-PSCA diabody linker between V-L and V-H domains
SGGGGS